(19) United States
(12) Patent Application Publication
(10) Pub. No.: US 2009/0205407 A1
(43) Pub. Date: Aug. 20, 2009

(54) MACHINE FOR TESTING THE BREATH ALCOHOL (ETHANOL) CONTENT OF PERSONS HAVING DRUNK ALCOHOLIC BEVERAGES

(75) Inventors: John Scott Marhefka, Mansfield, OH (US); David Michael Radomski, Butler, OH (US)

Correspondence Address: GREER, BURNS & CRAIN, 300 S WACKER DR, 25TH FLOOR, CHICAGO, IL, 60606, US

(73) Assignee: National Patent Analytical Systems, Inc., Mansfield, OH (US)

(21) Appl. No.: 12/399,139

(22) Filed: Mar. 6, 2009

Related U.S. Application Data
(62) Division of application No. 10/858,090, filed on Jun. 1, 2004, now abandoned.

(51) Int. Cl. G01N 33/497

(52) U.S. Cl. 73/23.3

(57) ABSTRACT

A method for testing the breath alcohol (ethanol) content with an embedded PC computer with a touch screen display uses software graphics with icons and graphs displaying an instant representation of the flow rate and breath alcohol concentration of the breath being delivered by the person taking the test. A software graphics using icons and graphs provides control panels by which a test administrator or technician is able to control fundamental operations and adjustments of the instrument. A geared, stepped, multiple optical component placement system having dual plates retains these components, and is capable of precision placement of all components using one electro-mechanical device. An infrared optical filter system operates between 3 and 10 microns. The results are stored in memory and are capable of comparisons to empirical tables for chemical identification.

This is a divisional application of pending application Ser. No. 10/858,090, filed Jun. 1, 2004.

### TECHNICAL FIELD

This invention relates generally to the field of evidential breath alcohol testing and more specifically to a machine for testing the breath alcohol (ethanol) content of persons having drunk alcoholic beverages.

### BACKGROUND OF THE INVENTION

Instruments measuring the amount of alcohol (ethanol is usually the form that is consumed) in human breath samples have been in widespread use for over 50 years. The results of the tests obtained from these instruments are used as evidence in court proceedings, particularly in Drunk Driving convictions with significant consequences to those defendants. The instruments are also used for alcohol related research and other areas. These instruments most often utilize either the principle of infrared absorption or that of electro-chemical fuel cell. The instrument in this invention utilizes infrared absorption although the concepts of the invention can apply to an electrochemical fuel cell.

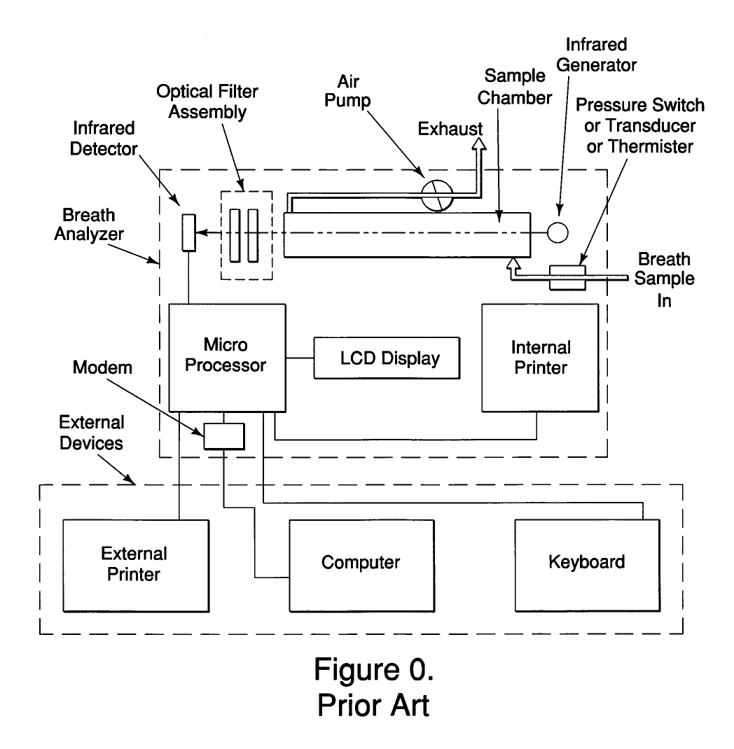

(See FIG. 0 for a general depiction of a typical breath analyzer). A typical alcohol breath analyzer includes a breath receiving mechanism into which the person whose breath alcohol level is to be measured exhales (blows) and a sample chamber receiver which retains a portion of the breath while an analysis is being performed. In the event the instrument operates using the principal of infrared absorption, there is also a source generator for the infrared signal, and an optical filter/detector system which performs the analysis by excluding all energy except those frequencies of infrared energy which have been shown to be absorbed by the alcohol molecules. The infrared energy that passes through the sample cell in the absence of alcohol produces a given level of signal from the infrared detector that forms the base line for the subsequent analysis. This is usually referred to as “zero”. When the alcohol laden breath sample is introduced into the sample chamber receiver, the amount of energy is lessened in accordance with the (*)Beer-Lambert law and can be compared to the base line “zero.” The difference between the base line signal and the analysis signal is quantified into an alcohol value reflected as a numerical value on an LCD type display. If the instrument operates using the principal of an electro-chemical fuel cell, the alcohol in the sample receiver reacts chemically and causes a flow of electrons that is proportional to the amount of alcohol present. This electrical output is converted to a numerical value and indicated as a number on the LCD display reflecting the results of the test.

### *Beer-Lambert Law:

There is an inverse relationship between the amount of chemical vapor present and the amount of infrared energy remaining after passing through the sample chamber in which the vapor resides.

Optionally, a keyboard is used during the testing process to enter information about the person being tested, along with other pertinent data. This information is stored in the instrument memory during the test and is then printed, along with the numerical test result at the end of the test to either an internal or a standalone external printer. If the test data is semi-permanently stored, it is stored in the memory of the instrument, and can be down loaded into a standalone external computer. The breath test instrument is typically contacted by the external PC Computer using a standard telephone modem. This is necessary, as most alcohol breath test instruments cannot, on their own, initiate the communication routine.

In all known breath test technologies, there is a pump used to purge the air system, a one way valve to prevent sucking backwards and adjustment devices used to adjust voltage settings and assure the proper operation of the instrument. There are also various methods of calibrating the instrument to assure that it is measuring alcohol values correctly including wet bath simulators containing a known amount of alcohol in water, dry gas standards containing a known amount of alcohol in an inert gas, quartz attenuation lens and other, less often used, methods.

All breath analysis instruments depend on a reasonably close sample of deep lung (alveolar) air because the science and research has shown that best approximation to blood alcohol is that of alveolar air. Alveolar air is reached only during the latter portion of the exhalation of the person being tested and it is therefore important that some controls be in place so as to assure that the breath delivery of the person being tested is sufficient in continuity and duration to insure that this alveolar sample is reached. First, sensing of the breath flow is done by utilizing either a thermistor that is sensitive to temperature change, or by use of a pressure switch or pressure transducer, all of which are placed either in or adjacent to the breath path. Each of these methods offer varying degrees of assurance that adequate flows are being obtained during the blowing of the breath. Thus assured that a sufficient breath flow is being obtained, a further control to insure that the sample of breath at the time of analysis is alveolar is often used. This is most commonly done by electronically measuring the rate of increase of the alcohol in the breath sample and inhibiting completion of the test until the characteristic uniformity of concentration typical of alveolar air is measured. Another, but more rudimentary, method used is simply by requiring a specific time of blow. Some instruments simply leave the time of blow to the discretion of the person administering the test.

It is also important that there be some method of insuring that no residual alcohol from a recently ingested alcoholic beverage is present in the mouth during the test that could compromise the results of the analysis. This is done primarily by requiring that there be a minimum 15 minute observation/deprivation period during which no alcohol is introduced into the mouth of the person being tested but there is also typically a further control as follows: It is well accepted by the scientific communities working with alcohol breath testing that the alcohol concentration of normal sample of human breath will rise as a person blows into the instrument. If the sample is delivered continuously and there are no other anomalies present, there is no reason why the concentration of alcohol in the breath should ever diminish during any given exhalation. If the analytical technique is that of infrared absorption, a method of computer monitoring of the rate of increase of alcohol is done to insure that the alcohol concentration of the breath sample rises throughout the sample delivery, Since one of the characteristics of mouth alcohol is a very rapid dissipation of the alcohol from the mouth area typically resulting in a high reading followed by a lower reading, the use of this method of detecting a diminishing alcohol concentration value enables these instruments to invalidate questionable tests.

Persons taking an alcohol breath test often make various attempts to defeat the testing instrument. These include sucking instead of blowing, blowing out the side of the breath receiving apparatus and blowing in a generally discontinuous manner. These efforts at defeat are interpreted by the instrument and result in an instrument condition that will terminate the test. The test administrator is advised that the test is an “invalid test” or some other indication given that there was a problem with the sample delivery. The test administrator is also advised by a short message on the LCD display.

All alcohol breath analysis instruments require periodic maintenance. Traditionally, this maintenance has been done using various measuring devices such as a volt-ohm meter, an oscilloscope or other devices while probing the internal electrical measurement points and manually adjusting potentiometers with screwdrivers to obtain the proper voltage settings.

Many administering agencies have begun utilizing outside computer systems to communicate with the se instruments in order to retrieve the test data for breath, calibration and diagnostic tests for use in quality assurance control programs. These agencies then statistically analyze the data for program control, court testimony and for dissemination to other interested safety agencies such as NHTSA. These agencies can also remotely conduct various quality assurance tests and monitor the condition of individual testing instruments using the phone modem capabilities. (See, as an example, http://www.sled.state.sc.us Link to “Implied Consent.”)

All administering agencies require through legally codified administrative rules, certain checklists, arrest or test forms, and multi language instructions for those who do not speak English. These forms are primarily executed manually.

“Specificity,” that is, the ability of the instrument to insure that only alcohol is present, is typical of most infrared breath analyzers, and reasonably inherent to electro-chemical fuel cell analyzers (except for alcohols other than ethanol). Infrared analysis typically uses two or more optical filters to accomplish this task of specificity. Since the absorption of ethanol is fixed and known at these two filter wavelengths, the expected ratio of the two optical filters to each other can be calculated and stored in memory or compared in some other fashion. Each subsequent test sample is then compared against this expected ratio. If a chemical other than ethanol is present in the breath sample, it will not produce the same ratio as the one that is known for ethanol. When this condition is detected by the instrument, the test is typically terminated with an appropriate message displayed. Currently, some enforcement agencies use video to provide a permanent record of the test, taping the person being tested through the proceedings, along with the actual breath test.

All breath testing instruments prior to this instrument have been controlled devices, that is, essentially being controlled either by a limited micro processor integral to itself, or by accepting commands from a computer external to itself.

### DEFICIENCIES OF THE CURRENT ART

The memory that can be allocated to data storage in these instruments is somewhat limited, de pending on how much information is entered for each test. Typically, the capacity of an alcohol breath test instrument is not greater than 100 tests, assuming the information entered is minimal.

None of the methods of insuring the delivery of alveolar air are completely without fault, as each leaves varying degrees of doubt as to the true quality of alveolar air that was really tested.

Although detecting a diminishing alcohol value is generally thought to be a reasonably effective method of eliminating the effects of “mouth alcohol,” it is occasionally heard in court from defense counsel that there can be conditions where these tools may not be entirely effective, such as “burps.” With only a numerical value displayed for a test result, there is limited information available to the operator with which to evaluate the rise in alcohol concentration and there is no way to visually confirm the presence or absence of “mouth alcohol.”

Efforts on the part of the person being tested to defeat the instrument mayor may not be obvious to the test administrator and often are interpreted by the test administrator as a refusal to take the test due to uncooperative behavior on the part of the person being tested. This often results in additional criminal penalties. Since only the subjective observation of the officer administering the test is available to substantiate the “refusal” behavior the whole matter typically becomes subject to sometimes extensive court proceedings during which many different possible causes of the “invalid sample” can be called into question. Some of these are instrument malfunctions, test administrator errors and the possible inability of the person to blow due to some physical condition. A more definitive answer to this condition is needed so that these questions can be eliminated in courts.

The maintenance process is often quite lengthy, tedious, training intensive and always involves removal of covers and sometimes even disassembly of the instrument for routine adjustments.

While some instruments provide limited written instructional prompts on the display, none provide voice prompts, or significant instructive text strings. Further, none have addressed the inefficiency of the manual execution of forms.

The question of possible other chemicals causing an overstatement of the alcohol test has always been an underlying issue. With only a single set of ratio relationships calculated and stored, no breath analyzers have the ability to determine the kind of chemical present if something other than alcohol (ethanol) is detected. Consequently, when the instrument determines that something other than alcohol is present, there remains an open question as to exactly what may have been causing the condition. Some of these possible causes can be instrument anomalies, sample delivery inconsistencies or a human related condition where an interfering compound is actually present.

Video tapes are separately cataloged and stored until needed, a tedious and time consuming process.

No breath test devices have the capability to control other devices, excepting a single printer, or act as a controller simultaneously to several other devices, or to be controlled while controlling other devices.

### BRIEF SUMMARY OF THE INVENTION

Therefore, it is the object of this invention to provide a breath alcohol measuring instrument that utilizes an embedded PC computer with a graphics touch screen display allowing the user to execute commands by touching a button on the display (icons). This is totally novel to an alcohol breath test instrument.

Another object is to provide a breath alcohol measuring instrument that utilizes an embedded PC computer with a graphics touch screen display and an air flow sensor allowing the electronic interpretation of both the rate and direction of the air flow so that the data presented to the computer for interpretation can be used in the following objectives, which are totally novel to a alcohol breath test instrument.

Another object is to provide a breath alcohol measuring instrument that utilizes an embedded PC computer with a graphics touch screen display allowing the test administrator to monitor a visual and graphical representation of the breath flow of the person being tested as the breath sample is being delivered, greatly enhancing the quality of the information being presented to the test administrator pertinent to the perceived cooperation of the person being tested.

Another object is to provide a breath alcohol measuring instrument that utilizes an embedded PC computer with a graphics touch screen display that stores in memory and prints the data integral to the breath flow graph for later use in court proceedings and quality assurance control. This is a significant advance in the quality of information that can be presented to judges and juries.

Another object is to provide a breath alcohol measuring instrument that utilizes an embedded PC computer with a graphics touch screen display allowing the test administrator to monitor a visual and graphical representation of the increase in alcohol concentration of the person being tested as the breath sample is being delivered so that the data presented to the computer for interpretation can be used in the following objectives, which are totally novel to an alcohol breath test instrument:

Another object is to provide a breath alcohol measuring instrument that utilizes an embedded PC computer with a graphics touch screen display allowing the operator to monitor a visual and graphical representation of the increase in alcohol concentration of the person being tested as the breath sample is being delivered, greatly enhancing the quality of the information presented to the trained test administrator regarding the possibility of “burps” or any other abnormal breath delivery conditions.

Another object is to provide a breath alcohol measuring instrument that utilizes an embedded PC computer with a graphics touch screen display that stores in memory and prints the data integral to the alcohol concentration graph for later use in court proceedings and quality assurance control. This is a significant advance in the quality of information that can be presented to judges and juries.

Another object is to provide a breath alcohol measuring instrument that utilizes an embedded PC computer with a graphics touch screen display that displays on the computer screen the voltages free of the necessity of opening the instrument and free of the use of external measuring devices. This is a very significant improvement in the ease and efficiency of the maintenance of these instruments.

Another object is to provide a breath alcohol measuring instrument that utilizes an embedded PC computer with a graphics touch screen display allowing the user to execute commands by touching a verbal or pictorial button on the display that is representative of the voltage adjustments and to change these voltages and adjustments in a manner that is free of opening the instrument and free of using any other devices typically used for adjustments. This is a very significant improvement in the ease and efficiency of maintenance of these instruments.

Another object is to provide a breath alcohol measuring instrument that utilizes an embedded PC computer with a graphics touch screen display allowing the user to retrieve pre-stored forms, checklists and multilingual instruction sets and display them on the graphics display. These forms can then be either read, if required, or completed by entering the appropriate information and then printed to either the internal or the external printer. This is a very significant improvement in the ease and efficiency of the operation of these instruments.

Another object is to provide a breath alcohol measuring instrument that utilizes an embedded PC computer with a graphics touch screen display that can accept a video input and provide a permanent electronic video record of the breath test on a computer disc. This is a very significant improvement in the efficiency of the operation of these instruments and programs and the ease with which the information can be presented to the courts.

Another object is to provide a breath alcohol measuring instrument that utilizes an embedded PC computer with a graphics touch screen display and instructional voice prompts to assist the test administrator in conducting the test. This is a very significant improvement in the efficiency of the operation of these instruments.

Another object is to provide a breath alcohol measuring instrument that utilizes an embedded PC computer with a graphics touch screen display that can simultaneously serve as a controlling or controlled device that connects to other peripherals singly or simultaneously for the purposes of data entry, data storage, data output, printing and automatic cellular digital connectivity to other systems. This will be a very significant advancement as the existing alcohol management programs continue to improve and expand their information gathering and sharing capabilities.

Another object is to provide a breath alcohol measuring instrument that utilizes an infrared optical filtration I calibration device controlled by a computer using dual wheels containing multiple optical components and operated by a single driver motor capable of precision placement of these components into the optical path, in any combination. This is totally novel to an alcohol breath test instrument. Further providing a breath alcohol measuring instrument that utilizes an infrared optical filtration I calibration device controlled by a computer using dual wheels containing multiple optical components and operated by a single driver motor capable of precision placement of these components into the optical path, in any combination, their position being sensed by optical sensors. This is totally novel to an alcohol breath test instrument.

Another object is to provide a breath alcohol measuring instrument that utilizes an embedded PC computer with a graphics touch screen display that utilizes a stored data base of absorption characteristics, empirically derived, to determine the kind of chemical present, if something other than alcohol is detected. This is a very significant advancement in the capabilities and operations of these instruments.

Other objects and advantages of the present invention will become apparent from the following descriptions, taken in connection with the accompanying drawings, wherein, by way of illustration and example, an embodiment of the present invention is disclosed.

In accordance with a preferred embodiment of the invention, there is disclosed an embedded PC computer operating with a touch screen graphics display.

In accordance with a preferred embodiment of the invention, there is disclosed an interconnected multiple optical component placement system, controlled by a computer and using more than one plate, each containing at least one optical component, capable of precision placement into an optical path, of any components on all plates, both independently and with respect to each other, using one electro-mechanical device.

In accordance with a preferred embodiment of the invention, there is disclosed an embodiment further comprising an infrared optical filter system operating between 3.3 and 10 microns, the results of which are stored in memory data banks and through the use of pre-tested empirical data and ratios, can be used by the imbedded PC to compare the instant test results and ratios against the stored data bank ratios and therefore identify the kinds of vaporous compounds, if any other than alcohol are present.

### BRIEF DESCRIPTION OF THE DRAWINGS

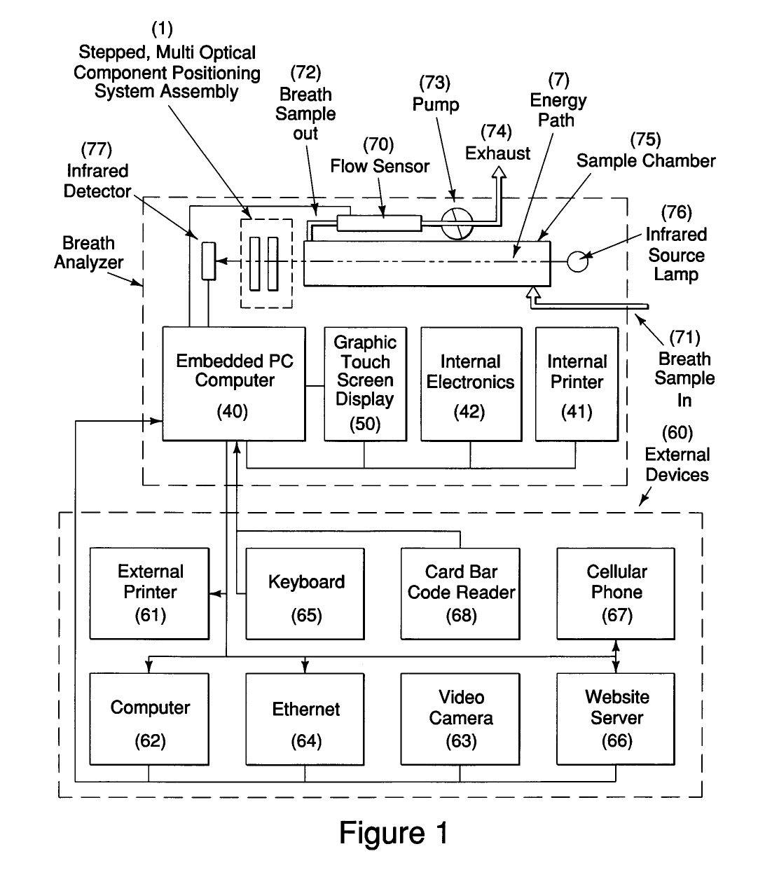

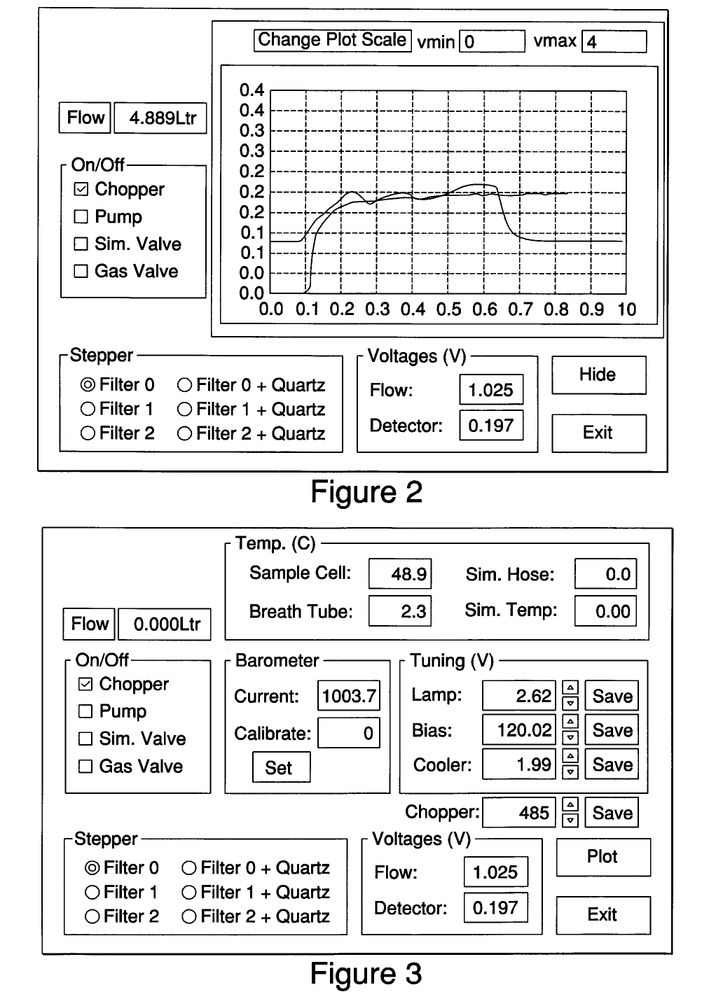

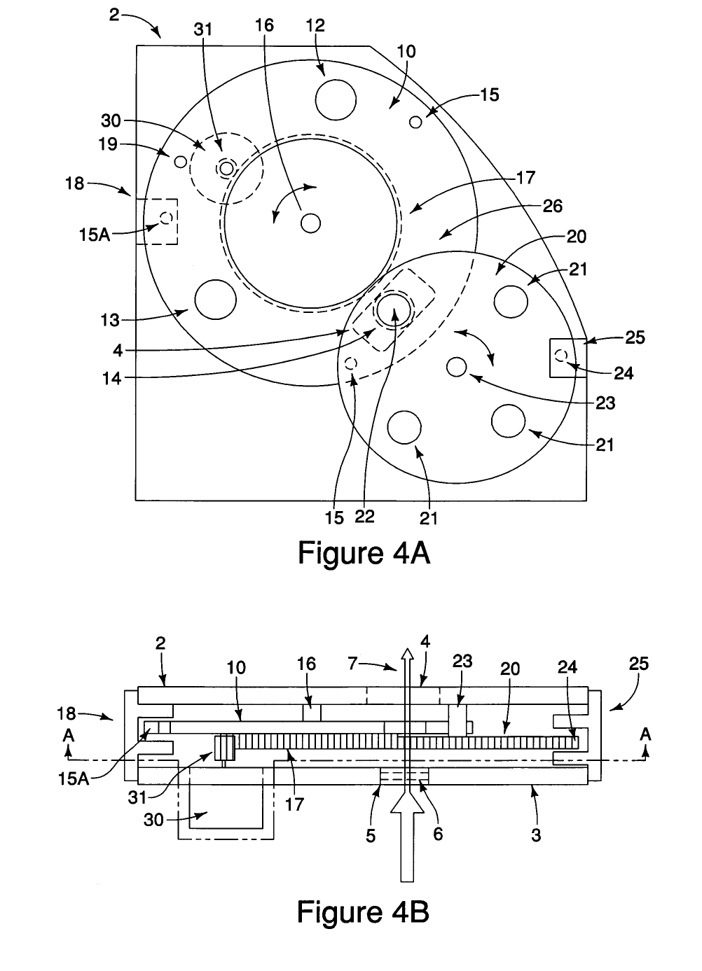

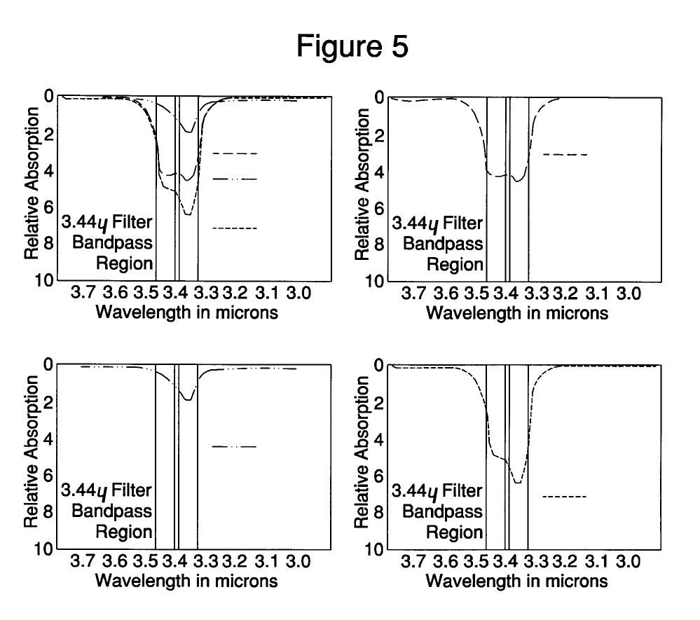

The drawings constitute a part of this specification and include exemplary embodiments to the invention, which may be embodied in various forms. It is to be understood that in some instances various aspects of the invention may be shown exaggerated or enlarged to facilitate an understanding of the invention. FIG. 0 shows an overall diagram of a breath analyzer for historical perspective only. FIG. 1 shows the overall diagram of the invention, FIG. 2 shows the computer generated on screen graphs visible to the test administrator as the person delivers a breath sample, FIG. 3 shows one of several computer generated control panels and FIGS. 4A and 4B show a front and top view of the Stepped, Multi Optical Component Positioning System, according to an embodiment of the invention. FIG. 5 depicts the ratio relationship of absorption characteristics between selected optical filters.

### DETAILED DESCRIPTION OF THE PREFERRED EMBODIMENTS

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

This invention is turned on by an “on/off switch” and then all normal operations are initiated by touching the graphic touch screen display (50) from which all other operations are commenced either automatically or by touching additional icons or buttons on the display (50). Certain data, such as the name of the individual being tested is entered either through the simulated keyboard (not shown) on the graphic touch screen display (50) or, optionally, through a traditional keyboard (65). All data being entered is displayed on the display (50) as it is entered.

References in this section are to FIG. 1 unless noted otherwise.

In this invention the infrared energy in the energy path (7) is produced by the infrared source lamp (76), passes through the sample chamber (75), exits the sample chamber (75), and is focused by the focusing lens (FIG. 4B (6)) residing in aperture (FIG. 4B, (5)) of the front retaining plate (FIG. 4B, (3)) and enters the stepped, multi optical component placement system (1). It is at this point where the Infrared Energy is filtered by the infrared optical filters (FIG. 4A, (12, 13 and 14)), allowing only the wavelengths of analytical interest to pass through. The infrared energy is then sensed by the detector (77) and an electrical signal is produced in an inverse proportion to the amount of energy present. A blank or “zero” base line is established by the computer (40) based on the output of the detector (77) before the sample is introduced into the sample chamber (75). The sample is introduced at the breath sample “in” point (71) and flows through the sample chamber (75) through which the infrared energy is passed. The breath then exits at exhaust (74) passing the breath sample out point (72) and the flow sensor (70) which determines electronically how much breath is flowing. The pump (73) is used to purge any test remnants and pull ambient air into the sample chamber (75). The beginning base line is then compared to the amount of energy remaining after the sample is introduced and the Infrared Energy has passed through it. The difference is quantified by the computer (40) according to the Beer-Lambert law. This difference is processed by the embedded PC computer (40) from which a number of things transpire. Among them are:

During the introduction of the sample, an electrical signal, proportional to the flow of air that is passing across the flow sensor (70) is processed by the embedded PC computer (40) and displayed on a graph (FIG. 2) on the graphic touch screen display (50) visible to the test administrator. This is very advantageous in determining the cooperation level of the person being tested since intentional variations in the blowing efforts of persons being tested will be immediately reflected by variations on the flow graph. The information comprising this graph is then stored in the memory of the embedded PC computer (40) and can be printed or otherwise transmitted to other internal or external devices (41, 61, 62, 64, 66, 67).

Also during the introduction of the sample the electrical signal that is being generated from the detector (77) that is inversely proportional with the ethanol concentration of the breath sample being entered is processed by the embedded PC computer (40) and displayed on a graph (FIG. 2) on the graphic touch screen display (50) visible to the test administrator of the instrument. This is very advantageous in determining that there be a uniform and consistent rise in the alcohol concentration since a discontinuous rise is indicative of possible sample or delivery problems to a trained test administrator. The information comprising this graph is then stored in the memory of the embedded PC computer (40) and can be printed or otherwise transmitted to other internal or external devices (41, 61, 62, 64, 66, 67). At the completion of the testing sequences the test results and other information is displayed on the graphic touch screen display (50), including a simple numerical value of the test result, and all the data generated is stored in the imbedded PC computer (40) and printed through either the internal or external printers (41, 61). It may be further sent or transmitted to an external computer (s) (62), the Ethernet (64), a web site (66) or via cellular transmission (67) to other receiving devices.

In the normal course of an instrument's life it is necessary to periodically view, check and adjust various voltages, electrical setting and calibrations. In this invention, these voltages and settings are viewable on a control panel (FIG. 3) on the graphic touch screen display (50) accessed by touching an icon. This control panel interacts through the embedded PC computer (40) to the instrument electronics (42) and by the use of digital potentiometers allows the changing of voltages and settings by touching an up or down arrow on the control panel (FIG. 3) then pressing the icon named “save.” These settings are then changed and electronically stored permanently in the memory of the device, Other similar control panels are used in much the same fashion to change and save other less frequently used settings and options.

All references below are FIGS. 4A and 4B unless otherwise noted.

This portion of the description will set forth, in detail, Stepped, Multi Optical Component Positioning System FIGS. 4A and 4B and specifically the measurement at the 3.44 micron optical component (14) simultaneously with the quartz attenuator (22) as being typical of the operation of the device.

The overall design of this embodiment is comprised of an Optical Filters Retaining Plate (10), a Quartz Standard retaining Plate (20) mounted together on a rear (2) retaining plate. These above components are interconnected to each other by a geared interlocking mechanism (17, 26) and to a gear drive (31) on a Stepper Motor (30) mounted on the front retaining plate (3) which is connected to and drives the optical filters retaining plate (10) through a geared motor hub (31). There are three optical components (12, 13 and 14) residing in the Optical Filters Plate (10) that rotates on a hub (16) fastened to the rear retaining plate (2). There are 3 open apertures (21) and one aperture containing the quartz attenuator (22) residing in the quartz standard attenuator plate (20) that rotates on a hub (23) fastened to the rear retaining plate (2).

The two rotating plates (10 and 20) are dimensioned such that when connected together by the geared interlocking mechanisms (17, 26), they are caused to be positioned relative to each other so that one of the three apertures (12,13 or 14) on the Optical filters Plate (10) will always align with one of four apertures (21 or 22) on the Quartz standard attenuator plate (20). Further, because of the difference in circumference between the two plates (10 and 20), one complete 360 degree rotation of plate (10) will cause a rotation of 270 degrees on plate (20). Since the four apertures (21 and 22) on plate (20) are spaced 90 degrees apart, a rotation of 270 degrees of plate (20) has the effect of decrementing the aperture position by one position, or 90 degrees (360 minus 270) when plate (10) is rotated clockwise as pictured in FIG. 4A.

Initially it is necessary for the embedded PC computer, (FIG. 1 (40)) to determine the position of the Optical filters Plate (10). Utilized for this purpose are 3 open apertures (2/15, 1/15A) and one open precursor aperture (19) on the Optical filters Plate (10) in conjunction with an optical position sensor (18) that determines when one of the positioning apertures (2/15, 1/15A or 19) permits the passage of light from one side of the positioning sensor (18) to the other side. The three open aperture (2/15, 1/15A) are fixed at 120 degrees apart, while the open precursor aperture (19) is positioned at a point that is 40 degrees prior to the open aperture (15A) that will cause the 3.44 micron filter (14) to be in the optical path (7). By computer counting the number of pulses required to turn the motor (30) therefore rotating the optical filters plate (10) to a position where, from one sensing of light to the next sensing of light, there is a shorter number of pulses equal to 40 degrees it Call be determined that the 3.44 optical filter (14) is now in the optical path (7).

Since one position causing the 3.44 micron filter (14) to be in the path (7) can coincide with any of four possible aperture positions of the quartz attenuator plate (20) also in the path, three with an open aperture (21) and one with the quartz aperture (22) it is further necessary to determine in which position the quartz attenuator plate (20) is located so that it can be moved, if necessary, to cause it to reside in the optical path at this time. This is accomplished as follows: A positioning aperture (24) is utilized on the quartz attenuator plate (20) in conjunction with an optical position sensor (25) that determines when positioning aperture (24) permits the passage of light from one side of the sensor (25) to the other. When light is sensed at the positioning aperture (24) it will always coincide with the quartz attenuator aperture (22) positioned in the optical path (7).

Beginning with the known position of the 3.44 optical filter (14) as it was described in the above paragraph where it becomes aligned in the optical path (7), the motor (30) rotates the Optical Filter Plate (10) with a predetermined number of pulses that will cause it to turn a full 360 degrees. Due to the ratio between wheels (10) and (20), this will cause wheel (20) to rotate 270 degrees, thus advancing it to the next decremented 90 degree position. If light is sensed at sensor (25) as caused by the alignment of positioning aperture (24) then it is known that both the 3.44 optical filter (14) and qml1iz standard (22) are in the optical path (7) at the same time. If light is not sensed, plate(10) is rotated in additional 360 degrees increments, decrementing the aperture (21 and 22) by 90 degrees or one position each time there is a 360 degree rotation of the Optical Filters Plate (10) until light is sensed at sensor (25) at which time it is known that both the 3.44 optical filter (14) and quartz standard (22) are in the optical path (7) at the same time.

This relative position enables a measurement to be taken by the detector (FIG. 1, (77)) while both the 3.44 micron filter (14) and the quartz attenuator (22) are residing in the energy path (7). Once this beginning position is known, any combination of placement of any given optical filter (12, 13, 14) into the optical path (7) either with the quartz attenuator (22) also in the path, or with an open aperture (21) in the path can be accomplished using a predetermined number of motor (30) pulses as stored in the memory of the embedded PC computer (40).

During the course of any given test it is necessary to measure the adsorption of infrared energy at more than 1 wavelength and in this instance, measurement is possible at 3 different wavelengths in addition to a calibration point These wavelengths are 3.37, 3.44 and 3.50 microns (12, 13 and 14), although it is common to use other wavelengths. Measurement of the infrared energy at multiple wavelengths permits the comparison of the adsorption characteristics of the energy at the different wavelengths and therefore the establishing of mathematical ratios of these characteristics. FIG. 5 describes their relationship between ethanol and acetone using a simpler system comprised of 2 filters.

Since ethanol has distinctive adsorption characteristic at each utilized frequency when analyzed alone, the ratio between these frequencies, as seen during the test, can be calculated and stored in memory. Further, since the ratios between the filter absorption is unique for each compound, and since the ratios between the filter absorption for combinations of compounds are unique, these ratios are readily determined by introducing known samples of the chemicals of interest into the sample chamber in vapor form, analyzing them, calculating the resulting ratios between the filters and storing these ratios in computer memory tables for later reference. The use of additional filters and the ratios inherent to them and each other infinitely expand the potential for identification of various compounds. The use of pre-stored empirical values and the ratios inherent to them to identify vaporous compounds in the human breath using an alcohol breath analyzer is unique and will be of significant value in courtroom proceedings where the defendant is claiming that vaporous compounds in the environment he was breathing (other than ethanol) were contributing to the reading.

While the invention has been described in connection with a preferred embodiment, it is not intended to limit the scope of the invention to the particular form set forth, but on the contrary, it is intended to cover such alternatives, modifications, and equivalents as may be included within the spirit and scope of the invention as defined by the appended claims.

What is claimed is:

1. A method for measuring the alcohol content of a human breath sample with an alcohol breath analysis instrument and a PC Computer, the method comprising:
producing a real-time graphics display of breath flow rate,
producing a real-time graphics display of ethanol concentration in the breath sample, and
displaying selected machine voltages and settings.

2. A method as set forth in claim 1 comprising:
displaying an electrical output of a flow sensor, in real time as a flow graph as processed by the PC Computer,
indicating the slope of the curve created by the blowing pattern of a person being tested, either positive or negative, at the same time that the person delivers the breath sample, and
recording this information in a computer memory.

3. A method as set forth in claim 1 comprising:
displaying in graphic form the electrical output of a detector, as processed by the PC Computer, indicating the breath alcohol concentration of a person being tested,
indicating the slope of the curve, either positive or negative, at the same time the person delivers the breath sample, and recording this information in a computer memory.

4. A method as set forth in claim 1, comprising:
operating an infrared optical filter system between 3.3 and 10 microns, storing the results in memory and through the use of pre tested and memory stored empirical data and ratios, using the PC Computer to validate and compare instant test results and ratios to those empirically measured and stored in memory, thus identifying the kinds of vaporous compounds present, if any are, other than alcohol.
